(19) United States
(12) Patent Application Publication
(10) Pub. No.: US 2026/0263015 A1
(43) Pub. Date: Sep. 10, 2026

(54) IMAGE DOMAIN MOTION COMPENSATION FOR HELICAL COMPUTED TOMOGRAPHY (CT)

(71) Applicant: GE Precision Healthcare LLC, Waukesha, WI (US)

(72) Inventors: Jed Pack, Glenville, NY (US); Pengwei Wu, Clifton Park, NY (US)

(73) Assignee: GE Precision Healthcare LLC, Waukesha, WI (US)

(21) Appl. No.: 19/070,433

(22) Filed: Mar. 4, 2025

(51) Int. Cl. A61B 6/03; A61B 6/02; G06T 3/18; G06T 7/11; G06T 7/20; G06T 11/00; G06T 15/00; G16H 30/20; G16H 30/40

(52) U.S. Cl. CPC A61B 6/032; A61B 6/027; G06T 3/18; G06T 7/11; G06T 7/20; G06T 12/20; G06T 15/00; G16H 30/20; G16H 30/40

(57) ABSTRACT

A computed tomography imaging system includes a data acquisition system configured to generate projection data indicative of X-ray radiation traversing a volume over a scan angular illumination range during a helical scan. The imaging system further includes a reconstructor configured to reconstruct the projection data based on a set of voxel-specific weighting functions to generate at least three sets of 3-D volumetric image data, each corresponding to a different virtual time point in the helical scan. The imaging system further includes a 3-D motion vector field determiner configured to determine a 3-D motion vector field between 3-D volumetric image data corresponding to a predetermined sub-set of the different virtual time points. The imaging system further includes a 3-D motion compensator configured to process the at least three sets of 3-D volumetric image data based on the 3-D motion vector field to generate 3-D motion compensated 3-D volumetric image data.

### FIELD

The following generally relates to computed tomography (CT), and more particularly to image domain motion compensation for helical CT.

### BACKGROUND

During a helical computed tomography (CT) scan, an X-ray source rotates around a subject supported in a bore while the subject is advanced through the bore, the X-ray source emits X-ray radiation that traverses the subject, and a detector array detects X-ray radiation that traverses the subject and impinges the detector array, a data acquisition system generates projection data indicative of the detected X-ray radiation, and a reconstructor reconstructs the projection data to generate three-dimensional (3-D) volumetric image data. Voxels of the 3-D volumetric image data are displayed as a two-dimensional (2-D) and/or the 3-D image using gray scale values corresponding to a relative radiodensity.

Subject motion during the helical CT scan may manifest as visible artifacts in the reconstructed 3-D volumetric image data. Examples of subject motion include voluntary (i.e., conscious) motion such as intentional movement of a part of the body (e.g., the head, an arm, a leg, etc.) and all of the body, etc., and/or involuntary motion such as movement of parts of the body in response to cardiac motion, respiratory motion, coughing, sneezing, etc. Examples of motion-based image artifact include shading, streaking, shape distortion, blurring, etc., which may degrade the image quality of the reconstructed 3-D volumetric image data, e.g., such that the image quality of the reconstructed 3-D volumetric image data is no longer or not diagnostic image quality.

In such instances, the subject may need to be scanned again. However, rescanning a subject increases overall X-ray radiation exposure to the subject for completing the ordered imaging examination, and X-ray radiation is ionizing radiation, which can damage and/or kill cells. Furthermore, rescanning the subject consumes X-ray technologist time and removes the imaging system as an available resource, and both could otherwise be utilized for scanning other patients. Existing approaches that address motion-based image artifact in helical CT scans require additional and/or repeated reconstruction steps and/or utilize deep-learning algorithms. Unfortunately, the former increase reconstruction time and/or require additional processing resources, and the latter is also susceptible to lack of explainability and/or generalizability.

In view of at least the foregoing, there is an unresolved need for another motion-based image artifact compensation approach for helical CT that mitigates at least the above-noted issues and/or shortcomings.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a computed tomography imaging system includes a data acquisition system configured to generate projection data indicative of X-ray radiation traversing a volume over a scan angular illumination range during a helical scan. The computed tomography imaging system further includes a reconstructor configured to reconstruct the projection data based on a set of voxel-specific weighting functions to generate at least three sets of 3-D volumetric image data, each of the at least three sets corresponding to a different virtual time point in the helical scan. The computed tomography imaging system further includes a 3-D motion vector field determiner configured to determine a 3-D motion vector field between 3-D volumetric image data corresponding to a predetermined sub-set of the different virtual time points. The computed tomography imaging system further includes a 3-D motion compensator configured to process the at least three sets of 3-D volumetric image data based on the 3-D motion vector field to generate 3-D motion compensated 3-D volumetric image data.

In another aspect, a computer-implemented method includes obtaining projection data indicative of X-ray radiation traversing a volume over a scan angular illumination range during a helical CT scan. The computer-implemented method further includes reconstructing the projection data based on a set of voxel-specific weighting functions to generate at least three sets of 3-D volumetric image data, each of the at least three sets corresponding to a different virtual time point in the helical scan. The computer-implemented method further includes determining a 3-D motion vector field between 3-D volumetric image data corresponding to a predetermined sub-set of the different time points. The computer-implemented method further includes processing the at least three sets of 3-D volumetric image data based on the 3-D motion vector field to generate 3-D motion compensated 3-D volumetric image data.

In another aspect, a computer readable medium is encoded with computer executable instructions. The computer executable instructions, when executed by a processor, cause the processor to obtain projection data indicative of X-ray radiation traversing a volume over a scan angular illumination range during a helical CT scan. The instructions further cause the processor to reconstruct the projection data based on a set of voxel-specific weighting functions to generate at least three sets of 3-D volumetric image data, each of the at least three sets corresponding to a different virtual time point in the helical scan. The instructions further cause the processor to determine a 3-D motion vector field between 3-D volumetric image data corresponding to a predetermined sub-set t of the different time points. The instructions further cause the processor to process the at least three sets of 3-D volumetric image data based on the 3-D motion vector field to generate 3-D motion compensated 3-D volumetric image data.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a non-limiting example of an imaging system configured for computed tomography (CT) imaging and including a subject motion compensation module, in accordance with an embodiment(s) herein.

FIG. 2 schematically illustrates a non-limiting example of the subject motion compensation module that includes temporal state view-weighting functions, a 3-D motion vector field determiner, and a 3-D motion compensator, in accordance with an embodiment(s) herein.

FIG. 3 graphically illustrates, in 2-D, an illumination start angle for a voxel of the scanned 3-D volume, in accordance with an embodiment(s) herein.

FIG. 4 graphically illustrates, in 2-D, an illumination end angle for the voxel discussed in connection with FIG. 3 along with an angular illumination range for the voxel, in accordance with an embodiment(s) herein.

FIG. 5 graphically illustrates, in 2-D, an illumination start angle for another voxel of the scanned 3-D volume, in accordance with an embodiment(s) herein.

FIG. 6 graphically illustrates, in 2-D, an illumination end angle for the other voxel discussed in connection with FIG. 5 along with an angular illumination range for the other voxel, in accordance with an embodiment(s) herein.

FIG. 7 schematically illustrates a non-limiting example of the 3-D motion vector field determiner discussed in connection with FIG. 2, in accordance with an embodiment(s) herein.

FIG. 8 graphically illustrates a non-limiting example of first, middle and last 180° angular ranges within an angular illumination range that is greater than 360°, in accordance with an embodiment(s) herein.

FIG. 9 graphically illustrates a non-limiting example of overlapping angular range between the first, the middle and the last 180° ranges within the angular illumination range discussed in connection with FIG. 8, in accordance with an embodiment(s) herein.

FIG. 10 graphically illustrates a non-limiting example of first, middle and last 180° ranges within an angular illumination range that is less than 360°, in accordance with an embodiment(s) herein.

FIG. 11 graphically illustrates a non-limiting example of overlapping angular range between the first, the middle and the last 180° ranges within the angular illumination range discussed in connection with FIG. 9, in accordance with an embodiment(s) herein.

FIG. 12 schematically illustrates a non-limiting example of the 3-D motion compensator discussed in connection with FIG. 2, in accordance with an embodiment(s) herein.

FIG. 13 schematically illustrates a non-limiting example of preparing sets of temporal-state 3-D volumetric image data for weighting with the 3-D motion compensator discussed in connection with FIG. 12, in accordance with an embodiment(s) herein.

FIG. 14 schematically illustrates a non-limiting example of a weighting function utilized by the 3-D motion compensator discussed in connection with FIG. 12, in accordance with an embodiment(s) herein.

FIG. 15 illustrates a non-limiting example of a flow chart for a computer-implemented method for generating motion compensated 3-D volumetric image data, in accordance with an embodiment(s) herein.

### DETAILED DESCRIPTION

Embodiments of the present disclosure will now be described, by way of example, with reference to the figures, in which a system, a method and/or instructions on a computer readable medium for image domain 3-D motion compensation of subject motion (e.g., such as voluntary and/or involuntary subject motion) in 3-D volumetric image data for helical CT scans. As discussed above, subject motion during a helical CT scan manifests as motion-based image artifact in the reconstructed 3-D volumetric image data, including shading, streaking, shape distortion, blurring, etc., which may degrade the image quality of the reconstructed 3-D volumetric image data, e.g., such that the image quality of the reconstructed 3-D volumetric image data is not or no longer diagnostic image quality.

Further discussed above, in such instances, the subject may need to be scanned again, but rescanning increases overall X-ray radiation dose to the subject for completing the ordered imaging examination, and X-ray radiation is ionizing radiation, which can damage and/or kill cells. Furthermore, rescanning consumes X-ray technologist time and removes the imaging system as an available resource, and both could be otherwise utilized for scanning other patients. Example existing approaches that address motion-based image artifact in helical CT scans have required additional and/or repeated reconstruction steps and/or utilized deep-learning algorithms. However, unfortunately, the former increases reconstruction time and/or requires additional processing resources, and the latter is also susceptible to lack of explainability and/or generalizability.

As described in greater detail below, with the approach herein multiple reconstructions are obtained. These reconstructions differ in terms of the view data range used to reconstruct each voxel. Each voxel in the reconstruction (image) domain projects onto the detector for a certain time range, which also corresponds to a certain view angle range since the view angles are acquired over time. The view angle range for any voxel is typically greater than 180 degrees, which is the minimum amount of data needed to produce an acceptable image in the neighborhood of that voxel. Consequently, different 180 degree subranges of the entire view angle range can be used to reconstruct different temporal states of the neighborhood of that voxel. A temporal mapping can be generated that takes as an input a particular spatial location and generates an output that specifies the time at the center of the temporal range corresponding to the views used to reconstruct that voxel. Generating multiple reconstructions as described above, each reconstruction would have a different temporal mapping.

For example, if a first reconstruction always uses the first available 180 degree range to reconstruct any given voxel and a second reconstruction always uses the last 180 degrees to reconstruct any given voxel, the temporal mapping of the first reconstruction at a particular voxel (A) will always produce a lower time than that of the second reconstruction at the same voxel (A). However, the time produced by the first reconstruction at that voxel (A) may be later (higher) than the time produced by the second mapping at a different voxel (B). Due to the helical nature of the acquisition, the time values produced by our temporal mapping will vary widely from one location to another in the image domain, indicating that the reconstruction as a whole is not representative of a single motion state (as it was in the neighborhood of a single voxel as noted above). Instead, the reconstructed image represents a continuously-varying patchwork of various motion states through time.

However, one could imagine a hypothetical motion state produced by placing each small piece (e.g., particle) of the scanned object at the location where it resided at the time indicated by the temporal mapping., Such a motion state is referred to herein as a “virtual temporal motion state,” representing a virtual time point, while in reality this motion state never occurred at any single time point. 3-D volumetric image data corresponding to at least a first virtual temporal motion state and a last virtual temporal motion state are utilized to estimate a 3-D motion vector field (MVF) for the voxels in the different sets of 3-D volumetric image data. The different sets of 3-D volumetric image data are processed according to the estimated 3-D MVF and recombined in the image domain to generate motion compensated 3-D volumetric image data. In one instance, the resulting 3-D volumetric image data provides a high-quality, high temporal-resolution CT images in a computationally-efficient manner, and/or mitigates shortcomings of existing approaches, as discussed herein.

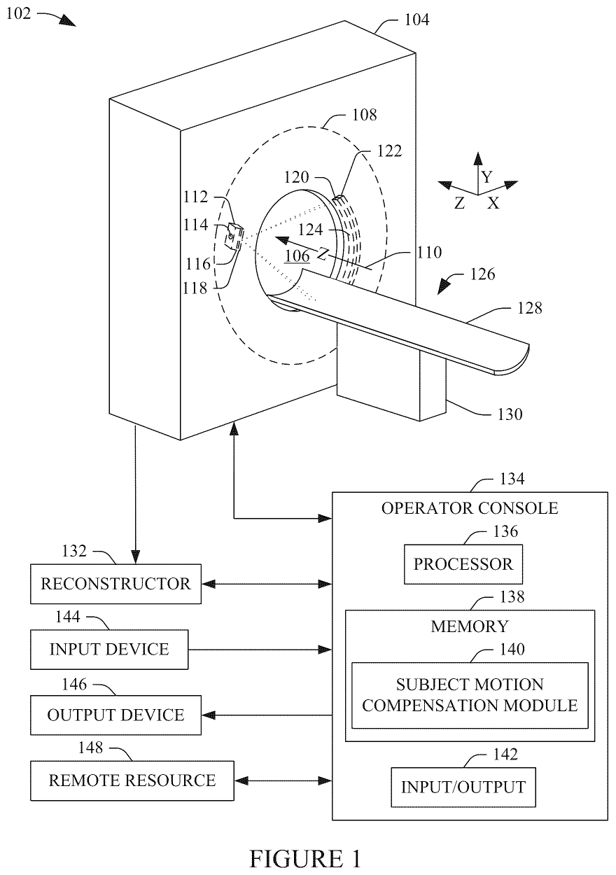

Initially referring to FIG. 1, a non-limiting example of an imaging system 102 configured for computed tomography (CT) imaging system is schematically illustrated. The imaging system 102 includes a gantry 104 including a bore 106 and a rotating frame 108. The rotating frame 108 is rotatably supported by the gantry 104, e.g., via a bearing (e.g., a slip ring, etc.) or the like, and is configured to rotate around the bore 106 about a rotational or z-axis 110, which extends through a center of rotation (e.g., a center of the bore 106, i.e., an isocenter). In some instances, the gantry 104 can be configured to tilt through the z-axis 110. A gantry controller is configured to control rotation (and tilt, if available) of the rotating frame 108, including no rotation.

The rotating frame 108 includes components utilized in the generation, emission and detection of X-rays. For instance, an X-ray source assembly 112 is supported by the rotating frame 108 and rotates in coordination with the rotating frame 108. The X-ray source assembly 112 includes an X-ray source 114 such as an X-ray tube and/or other source that generates and/or emits X-ray radiation. The X-ray assembly 112 further includes or is coupled to a filter 116, such as a filter that characterizes a radiation dose profile. The X-ray assembly 112 further includes or is coupled to a collimator 118 that shapes the X-ray radiation to form a generally fan, wedge, cone, etc. shaped beam that traverses the bore 106.

With a non-spectral configuration, the X-ray source 114 is configured to emit polychromatic broadband X-ray radiation having an energy at least in the X-ray diagnostic range such as twenty (20) kiloelectronvolts (keV) to one-hundred and fifty (150) keV), more, or less. With a spectral configuration, in one instance, the X-ray source 114 is configured to switch between at least two different kilovoltage peak (kVp) values and/or includes multiple X-ray tubes configured to emit in different spectrums (e.g., multiple mono-chromatic sources, etc.). An X-ray controller is configured to control components of the X-ray assembly 112, including the X-ray source 114 (e.g., such as kVp, etc.), the collimator 118 (e.g., a width of the X-ray beam), etc.

The components carried by the gantry 104 further include a detector array 120 and a Data Acquisition System (DAS) 122. The detector array 120 and the DAS 122 are supported by the rotating frame 108, opposite the X-ray source 114 along an arc, across the bore 106, and rotate in coordination with the rotating frame 108, including the X-ray source 114. The detector array 120 includes a one-dimensional (1-D) and/or a two-dimensional (2-D) array of rows of X-ray radiation sensitive detector elements 124. Each of the X-ray radiation sensitive detector elements 124 is in electrical communication with the DAS 122.

The X-ray radiation sensitive detector elements 124 include an indirect conversion detector such as a scintillator/photodiode detector and/or a direct conversion detector such as a Cadmium Telluride (CdTe), a Cadmium Zinc Telluride (CZT), etc. detector. Where the imaging system 102 includes a spectral configuration, in one instance, the detector array 120 includes multiple layers of detection materials, each array configured to detect a different energy, etc., where the projection data can be decomposed into photoelectric effect, Compton scattering, etc. material basis components. Additionally, or alternatively, the detector array 120 includes photon counting detectors that directly detect photon energy level. A DAS controller (not visible) controls the detector array 120, the X-ray radiation sensitive detector elements 124, etc.

The imaging system 102 includes a table 126. The table 126 includes a cradle 128 moveably coupled to a frame/base 130. In one instance, the cradle 128 is slidably coupled to the frame/base 130 via a bearing or the like, and a drive system (not visible) including a motor, a lead screw, and a nut (or other drive system) translates the cradle 128 along the frame/base 130 into and out of the bore 106 for horizontal motion, and the frame/base 130 includes a drive system (not visible) including a mechanism for vertical or diagonal motion. The cradle 128 is configured to support a subject in the bore 106 for loading, scanning, and/or unloading. A table controller (not visible) controls the drive system of the table 126.

For an axial (step and shoot) CT scan, the cradle 128 is positioned at a static position for each integration period and moves between integration periods, detecting X-ray radiation for one or more slices, which collectively form a volume. For a helical CT scan, the X-ray source 114 rotates and emits X-ray radiation at predetermined angular increments as the table 126 moves the subject along the Z-axis 110. For a helical CT scan, X-rays pass through each voxel of the scanned 3-D volume for only a sub-portion of a Z-axis extent of the helical CT scan. The X-rays passing through a voxel interact with tissues within the voxel and are attenuated by the tissues depending on density and composition of the tissues. Views of attenuated X-rays for a voxel are detected by the X-ray sensitive detectors 124 each integration period for the angular increments in which X-rays pass through the voxel. The DAC 122 generates projection data (line integrals) for each view that is indicative of the detected X-ray radiation.

Each voxel has a unique set of an illumination start angle (i.e., a first view angle at which X-rays pass through the voxel), an illumination end angle (i.e., a last view angle at which X-rays pass through the voxel), and an angular illumination range between the start illumination angle and the end illumination angle. In the case where the voxel experiences interrupted illumination (i.e., it projects onto the detector during more than one disconnected view subset), the illumination start angle will be considered to be the first view angle in the largest contiguous subset of views for which it projects onto the detector. Likewise, the illumination end angle will be considered as the last view angle in the same subset of views. This interrupted illumination condition can occur for some voxels when the helical pitch is relatively small. Metadata (e.g., a file header, etc.) is populated with sufficient information (e.g., scan geometry and image geometry specifiers) to calculate (for every voxel) values such as the start illumination angle, the end illumination angle, a direction of rotation, etc. The angular illumination range for the voxel can be determined from the start illumination angle and the end illumination angle, and/or otherwise. The angular illumination range is voxel-dependent and can vary between voxels. By way of non-limiting example, angular illumination range for one voxel may be two-hundred and eighty degrees (280°) while an angular illumination range for another voxel in a same 3-D volume may be four-hundred and twenty degrees (420°). Other angular illumination ranges are contemplated herein.

A reconstructor 132 reconstructs the projection data and generates 3-D volumetric image data for a helical scan an. The 3-D volumetric image data and/or 2-D slices thereof can be visually presented, filmed, etc. The reconstructor 132 employs view-weighting functions for helical scanning, employing the illumination start angle, the illumination end angle, and the angular illumination range, as discussed in greater detail below. Examples of suitable reconstruction algorithms include filtered back projection (FBP), advanced statistical iterative reconstruction (ASIR), conjugate gradient (CG), maximum likelihood expectation maximization (MLEM), model-based iterative reconstruction (MBIR), deep learning-based reconstruction, and/or other reconstruction algorithm.

A computing system 134 serves as an operator console of the imaging system 102. The computing system 134 includes a computer, a workstation, server, etc. The computing system 134 includes a processor 136 such as a microprocessor (mP), a central processing unit (CPU), graphics processing unit (GPU), etc., and a computer readable medium 138 (“MEMORY”), which includes non-transitory medium and excludes transitory medium (signals, carrier waves, and the like). The computer readable medium/memory 138 at least includes a subject motion compensation module 140.

The subject motion compensation module 140 is configured to compensate for subject motion that occurs during a helical CT scan. As described in greater detail below, the approach herein generates multiple virtual temporal state reconstructions (i.e., sets of 3-D volumetric image data for different virtual time points of the helical CT scan), where the different sets of 3-D volumetric image data capture different virtual temporal motion states of the subject. 3-D volumetric image data corresponding to at least a first virtual temporal state and a last virtual temporal motion state are utilized to estimate a 3-D motion vector field (MVF) for the different sets of 3-D volumetric image data. The different sets of 3-D volumetric image data are warped according to the estimated 3-D MVF and recombined in the image domain to generate motion compensated 3-D volumetric image data.

As discussed herein, such motion can manifest as shading, streaking, shape distortion, blurring, etc. in the reconstructed 3-D volumetric image data, and such image artifact may degrade the image quality of the reconstructed 3-D volumetric image data, e.g., such that the image quality of the reconstructed 3-D volumetric image data is not or no longer diagnostic image quality, and existing approaches that address motion-based image artifact in helical CT scans have required additional and/or repeated reconstruction steps and/or utilize deep-learning algorithms, which increase reconstruction time, require additional processing resources, and/or are susceptible to lack of explainability and/or generalizability. In addition, approaches that iteratively determine the motion (e.g., based on optimizing image metrics) can be much faster if the motion compensation is done using image domain processing (as is done in the algorithm presented herein) as there is no need for multiple backprojection operations.

In one instance, the approach described mitigates additional helical CT scans and/or repeated CT reconstruction steps of existing approaches that address motion-based image artifact in helical CT scans, reducing patient dose, processing resource requirements, and/or saving time. The approach described also mitigates the lack of explainability and/or generalizability associated with existing approaches that address motion-based image artifact in helical CT scans with AI based algorithms such as deep-learning, etc. algorithms. In addition, the resulting 3-D volumetric image data provides a high-quality, high temporal-resolution CT images in a computationally efficient manner.

The computing system 134 further includes input/output (I/O) 142. The computing system 134 is in electrical communication with the reconstructor 132 through the I/O 142 and/or otherwise. An input device 144 includes a keyboard, mouse, touchscreen, microphone, etc. The input device 144 is in electrical communication with the computing system 134 through the I/O 142 and/or otherwise. An output device 146 includes a human readable device such as a display monitor or the like. The output device 146 is in electrical communication with the computing system 134 through the I/O 142 and/or otherwise.

A remote resource 148 includes one or more of a server, a workstation, a Radiology Information System (RIS), a Hospital Information System (HIS), an Electronic Medical Record (EMR), a Picture Archiving and Communication System (PACS) for storing information, a PACS further configured with image viewing and/or manipulating software, cloud resources with shared remote data storage and/or computing power including resources distributed over data centers, etc. The computing system 134 and the remote resource 148 are in communication via wired and/or wireless technologies. Such communication can be through Digital Imaging and Communications in Medicine (DICOM), Health Level Seven (HL7), etc. formats and protocols.

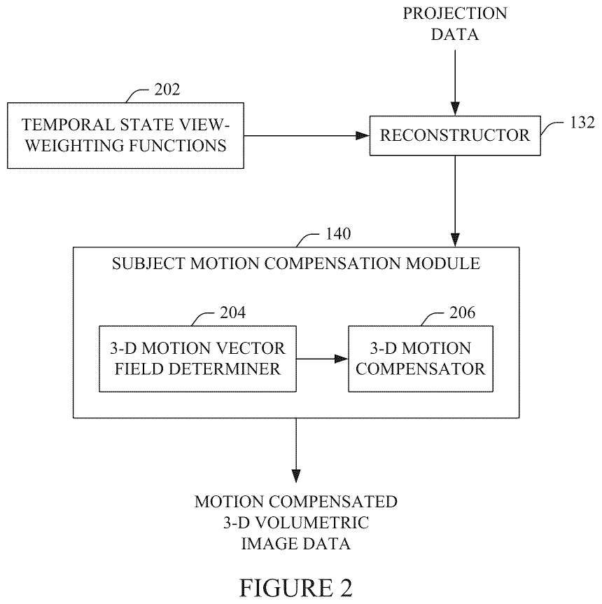

Turning to FIG. 2, a non-limiting example of the subject motion compensation module 140 is schematically illustrated in connection with the reconstructor 132 and temporal state view-weighting functions 202. The example subject motion compensation module 140 includes a 3-D motion vector field determiner 204, and a 3-D motion compensator 206. In this example, the reconstructor 132 described in connection with FIG. 1 receives the projection data and reconstructs at least three sets of temporal state 3-D volumetric image data based on the temporal state view-weighting functions 202, where the different sets of 3-D volumetric image data correspond to different virtual time points of the helical CT scan and capture different virtual temporal motion states of the subject.

The temporal state view-weighting functions 202 include multiple view-weighting functions for each voxel in each set of 3-D volumetric image data. In general, a helical CT scan acquires data over multiple revolutions, and X-rays pass through each voxel only for a sub-portion of the total angular range covered by the multiple revolutions of the helical CT scan. Each voxel will have a unique set of an illumination start angle at which X-rays first pass through the voxel, an illumination end angle at which X-rays last pass through the voxel, and an angular illumination range (the positive difference between the other two values) from the start angle to the end angle. As discussed herein, such information can be computed from metadata of the 3-D volumetric image data and/or otherwise.

The view-weighting functions for a particular voxel include at least a view-weighting function for a first one-hundred and eighty degrees (180°) of the angular illumination range for the voxel, a last view-weighting function for a last 180° of the angular illumination range for the voxel, and a middle view-weighting function for a middle 180° of the angular illumination range for the voxel. In another instance, the view-weighting functions for a particular voxel further include at least a view-weighting function for another 180° range of the angular illumination range for the voxel. Additionally, or alternatively, view-weighting functions can cover a different angular range, such as 10°, 12°, 15°, more, or less degrees of the angular illumination range for the voxel, with a number of such partial angles to cover 180° for at least a first, last and middle 180°.

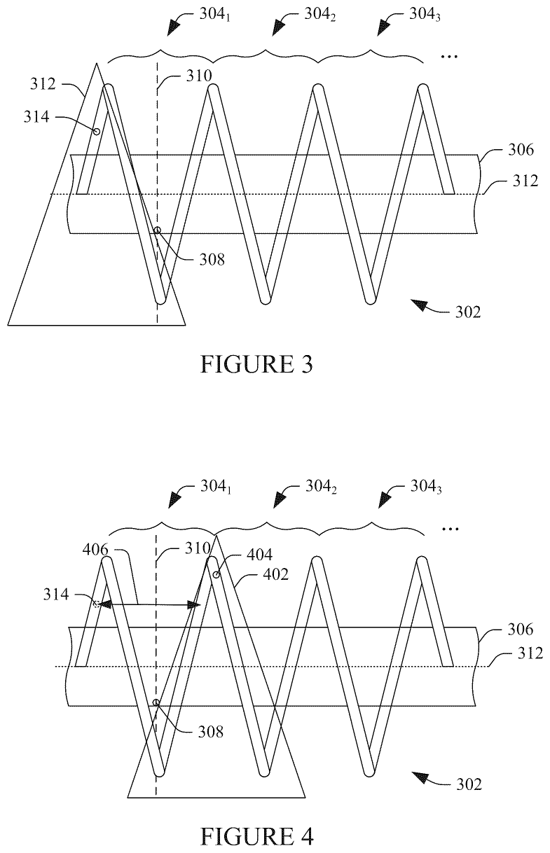

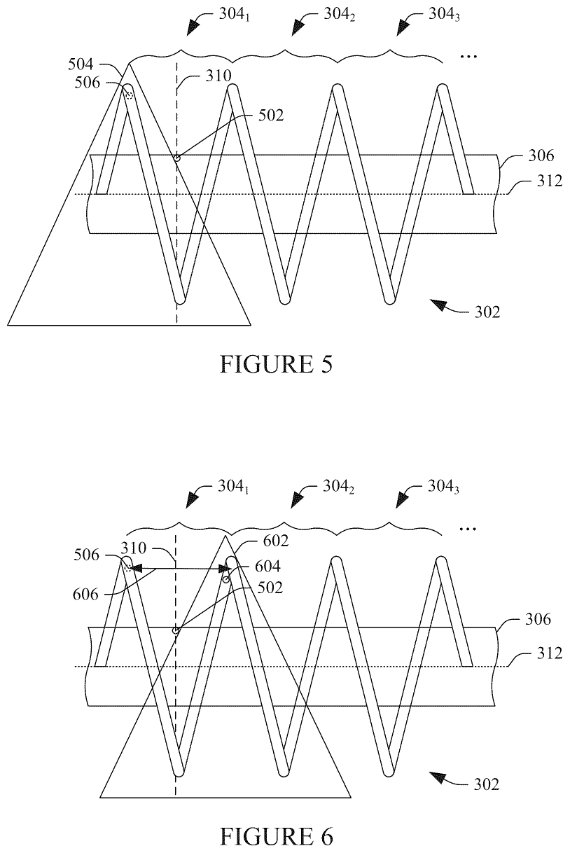

Again, the illumination start angle, the illumination end angle and the angular illumination range for a voxel depends on a location of the voxel within the 3-D volumetric image data. Examples of different sets of illumination start angles, illumination end angles and angular illumination ranges for different voxels in a same plane in the 3-D volumetric image data are graphically illustrated in FIGS. 3, 4, 5 and 6. For sake or clarity and explanatory purpose, the examples of FIGS. 3, 4, 5 and 6 are illustrated in 2-D. However, it is to be understood that the helical path is a 3-D path around a 3-D volume. FIGS. 3 and 4 illustrate illumination start and end angles for a voxel in one location in a plane of the 3-D volume, and FIGS. 5 and 6 illustrate illumination start and end angles for a voxel in different locations in the plane of the 3-D volume.

Initially referring to FIG. 3, a helical path 302 includes a plurality of revolutions 304₁, 304₂, 304₃, . . . around a 3-D volume 306. For a voxel 308 in a plane 310 of the 3-D volume 306, X-rays of a view 312 first traverse the voxel 308 at an angular position 314 (i.e., an illumination start angle) of the helical path 302 for the voxel 308. Moving to FIG. 4, X-rays of a view 402 last traverse the voxel 308 at an angular position 404 (i.e., an illumination end angle) of the helical path 302 for the voxel 308. An angular range 406 of the helical path 302 represents three-hundred and sixty degrees (360°). In this example, the angular position 404 is further than 360° away from the angular position 314. As such, the angular illumination range for the voxel 308 is greater than 360°.

Turning to FIG. 5, for a voxel 502 in the plane 310 of the 3-D volume 306, X-rays of a view 504 first traverse the voxel 502 at an angular position 506 (i.e., an illumination start angle) of the helical path 302 for the voxel 502. Moving to FIG. 6, X-rays of a view 602 last traverse the voxel 502 at an angular position 604 (i.e., an illumination end angle) of the helical path 302 for the voxel 502. An angular range 606 of the helical path 302 represents 360°. In this example, the angular position 604 is less than 360° away from the angular position 506. As such, the angular illumination range for the voxel 604 is less than 360°.

With references to FIGS. 2, 3, 4 5 and 6, other voxels in the plane 310 and/or other voxels in other planes will have a different set of illumination start angles, illumination end angles and angular illumination ranges, including a same and/or larger or smaller angular illumination ranges. In general, (at least when the helical pitch is close to 1.0) a voxel farther away from its illumination start angle will have a longer angular illumination range relative to a voxel closer to its illumination start angle. In FIGS. 3, 4, 5 and 6, the illustrated views 312, 402, 504 and 602 are all in a same direction, which is vertical in the drawings (with a center ray that is approximately perpendicular to a long axis 312 of the 3-D volume. However, it is to be understood that the illumination start and/or end angles can be horizonal with a center ray that is approximately parallel to the long axis 312 of the 3-D volume, oblique to the long axis 312, a combination thereof, etc.

With continuing reference to FIG. 2, in one instance, e.g., with a pitch of 1, the view-weighting functions for a particular voxel include at least a view-weighting function for a first one-hundred and eighty degrees (180°) of the angular illumination range for the voxel, a last view-weighting function for a last 180° of the angular illumination range for the voxel, and a middle view-weighting function for a middle 180° of the angular illumination range for the voxel. In this instance, the reconstructor 132 reconstructs first 3-D volumetric image data with the view-weighting function for the first 180°, last 3-D volumetric image data with the view-weighting function for last 180°, and middle 3-D volumetric image data with the view-weighting function for the middle 180°. 3-D volumetric image data can also be reconstructed for other angular ranges. For sake of brevity and explanatory purposes, a pitch of 1 is considered herein. However, other pitches are contemplated herein.

When the helical pitch is less than 1, the angular illumination range for at least one voxel can, in some instances, exceed 540° (e.g., when the pitch is 0.5, the angular illumination range for at least one voxel exceeds 540°), making it impossible to completely cover this range with three overlapping 180° sections (as described above) since the product of 3 times 180° is 540°. In this case, reconstruction of more than three initial volumes is required. For example, if the pitch is about 0.5, six initial volumes can be generated: one each from six different view weighting functions. The first still covers the first 180° of the illumination range and the last still covers the last 180°. However, the second through fifth angular ranges (each of which cover 180°) can be centered on angles that are, respectively, 180° before, 60° before, 60° after, and 180° after the central angle of the entire angular illumination range.

Continuing with reference to FIG. 2, the 3-D motion vector determiner 204 processes at least two of the at least three sets of 3-D volumetric image data and generates a 3-D motion field vector. For example, in one instance, the 3-D motion vector determiner 204 processes at least the first 3-D volumetric image data and the last 3-D volumetric image data to generate the 3-D motion field vector. In this instance, the first 3-D volumetric image data represent an initial virtual motion state (i.e., a reference no motion state) of the subject and the last 3-D volumetric image data represents a virtual motion state that represents subject motion that occurred during the helical CT scan from the illumination start angle to the illumination end angle of the angular illumination range.

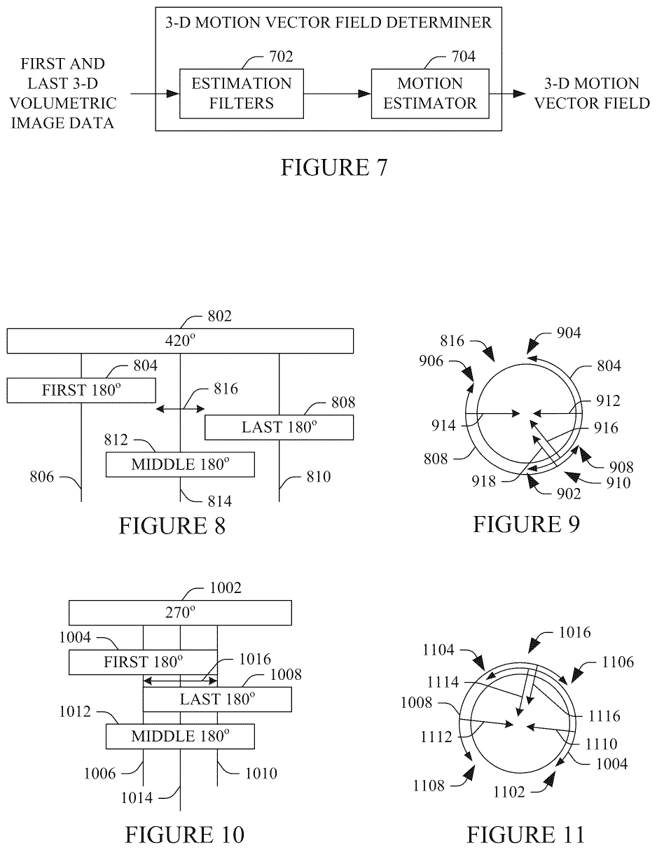

FIG. 7 schematically illustrates a non-limiting example of the 3-D motion vector determiner 204. The example the 3-D motion vector determiner 204 includes estimation filters 702 and a motion estimator 704. As discussed herein, the angular illumination range for a voxel can be less than 360°, equal to 360°, or greater than 360°. The estimation filters 702 are configured to filter out certain frequencies in the first 3-D volumetric image data and the last 3-D volumetric image data such that the angular separation (or delay) between the filtered first 3-D volumetric image data and the filtered last 3-D volumetric image data is equal to 180°. This filtering applies to the case mentioned above where the helical pitch is approximately 1.

In the case where the helical pitch is about 0.5, the second and fifth initial volumes are reconstructed from angular ranges that are already exactly 360° apart and are thus well suited for motion estimation without the need to apply pre filtration as described below (having the ranges separated by any integer multiple of 180° is the desired condition since it guarantees each spatial frequency has the same angular delay regardless of orientation). FIGS. 8 and 9 diagrammatically illustrate a scenario where the helical pitch is 1.0 and the angular illumination range is greater than 360°, and FIGS. 10 and 11 diagrammatically illustrate a scenario where the helical pitch is 1.0 and the angular illumination range is less than 360°.

Beginning with FIG. 8, in this example, an angular illumination range 802 for a particular voxel is 420°. A first angular range 804 represents an angular range of a first 180° of the 420° angular illumination range 802. The 3-D volumetric image data for the first angular range 804 is reconstructed at a center 806 of the first angular range 804/a first reconstruction time point. A last angular range 808 represents an angular range of a last 180° of the 420° angular illumination range 802. The 3-D volumetric image data for the last angular range 808 is reconstructed at a center 810 of the last angular range 808/a last reconstruction time point. A middle angular range 812 represents an angular range of a middle 180° of the 420° angular illumination range 802. The 3-D volumetric image data for the middle angular range 812 is reconstructed at a center 814 of the middle angular range 812/a middle reconstruction time point. Since the angular illumination range 802 is greater than 360° (i.e., 420°), an angular gap 816 exists between the first 180° angular range 804 and the last 180° angular range 808. In addition, the displacement between the first and last time points (806 and 810) is larger than the desired 180 degrees. This situation can be remedied by filtering.

Moving to FIG. 9, the first 180° angular range 804 and the last 180° angular range 808 are diagrammatically illustrated over 360°. In this example, the first 180° angular range 804 begins at a start angle 902 and ends at an end angle 904, and the last 180° angular range 808 begins at a start angle 906 and ends at an end angle 908. The last 180° angular range 808 begins after the angular gap 816 and overlaps the first 180° angular range 804 in an overlap region 910 when wrapped around (modulo 360) as shown. Pairs of rays 912 and 914 respectively from the first 180° angular range 804 and the second 180° angular range 808 are 180° apart. Pairs of rays 916 and 918 respectively from the first 180° angular range 804 and the second 180° angular range 808 are 360° apart. The estimation filter 702 removes all data corresponding to the rays in the overlap region 910 (i.e., duplicate rays), leaving only rays that are 180° apart, which can be cross-correlated to determine subject motion a half rotation apart.

Next at FIG. 10, in this example, an angular illumination range 1002 for a particular voxel is 270°. A first angular range 1004 represents an angular range of a first 180° of the 270° angular illumination range 1002. The 3-D volumetric image data for the first angular range 1004 is reconstructed at a center 1006 of the first angular range 1004/a first reconstruction time. A last angular range 1008 represents an angular range of a last 180° of the 270° angular illumination range 1002. The 3-D volumetric image data for the last angular range 1008 is reconstructed at a center 1010 of the last angular range 1008/a last reconstruction time. A middle angular range 1012 represents an angular range of a middle 180° of the 270° angular illumination range 1002. The 3-D volumetric image data for the middle angular range 1012 is reconstructed at a center 1014 of the middle angular range 1012/a middle reconstruction time. Since the angular illumination range 1002 is less than 360° (i.e., 270°), there is an overlap region 1016 of first 180° angular range 1004 and the last 180° angular range 1008. In addition, the displacement between the first and last time points (1006 and 1010) is smaller than the desired 180 degrees. This situation can be remedied by filtering.

Moving to FIG. 11, the first 180° angular range 1004 and the last 180° angular range 1008 are diagrammatically illustrated over 360°. In this example, the first 180° angular range 1004 begins at a start angle 1102 and ends 180° later at an end angle 1104, and the second 180° angular range 1008 begins at a start angle 1106 and ends 180° later at an end angle 1108. The second 180° angular range 1008 overlaps the first 180° angular range 1004 in the overlap region 1016. Pairs of rays 1110 and 1112 respectively from the first 180° angular range 1004 and the second 180° angular range 1008 are 180° apart. Pairs of rays 1114 and 1116 respectively from the first 180° angular range 1004 and the second 180° angular range 1008 are 0° apart. The estimation filter 702 removes all data corresponding to the rays in the overlap region 1016 (i.e., duplicate rays), leaving only rays that are 180° apart, which can be cross-correlated, or registered in another way to determine subject motion a half rotation apart.

Returning to FIG. 7, the estimation filters 702 are further configured to bandpass filter the remainder (i.e., after removing the overlap regions 910 and 1016) of the first 3-D volumetric image data and the second 3-D volumetric image data after removal of the overlapping regions. In one instance, the bandpass filter filters higher frequencies (e.g., noise, etc.) and/or lower frequencies (e.g., influences due to contrast, etc.) in the radial direction, and retains midrange frequencies for the motion estimation. Additionally, or alternatively, other approaches such as feature extraction like the Modality Independent Neighborhood Descriptor (MIND), which is a model for interpretable feature selection and extraction, a pre-defined network (e.g., nnUNet, etc.) that performs multi-organ/tissue segmentation, etc. can be utilized.

The motion estimator 704 is configured to estimate a 3-D motion vector field (MVF) between the filtered first 3-D volumetric image data and the filtered last 3-D volumetric image data for each voxel. Any known and/or other registration algorithm can be employed. In one instance, a registration approach is utilized. For example, in one instance the 3-D MVF is iteratively estimated through minimizing a difference between the filtered first 3-D volumetric image data (reference) and the filtered last 3-D volumetric image data (warped by the current 3D MVF). The motion estimator 704 divides the 3-D MVF by 180°to convert the 3-D MVF to units of mm/angle (i.e., motion speed). In one instance, the motion estimation algorithm is performed using a GPU-accelerated demons registration algorithm to find the 3-D MVF between the filtered first 3-D volumetric image data and the filtered last 3-D volumetric image data. An example of such an algorithm includes a multi-resolution Thirion's demons.

Another suitable algorithm includes a Sum of Squared Differences (SSD), which minimizes a sum of squared differences between intensities of the voxels in the filtered first 3-D volumetric image data and the filtered last 3-D volumetric image data. Another suitable algorithm includes a Symmetric Normalization (SyN), which uses a symmetric diffeomorphic transformation model. Another suitable algorithm includes a Large Deformation Diffeomorphic Metric Mapping (LDDMM). Another suitable algorithm includes a VoxelMorph, which is a learning-based approach for deformable registration that uses convolutional neural networks (CNNs) to learn a parametric function that maps input image pairs to a deformation field. In another instance, a four-dimensional (4-D) approach is utilized. For instance, the filtered first 3-D volumetric image data and the filtered last 3-D volumetric image data could be divided into four partial angle reconstructions and concatenated in 4-D before the registration steps.

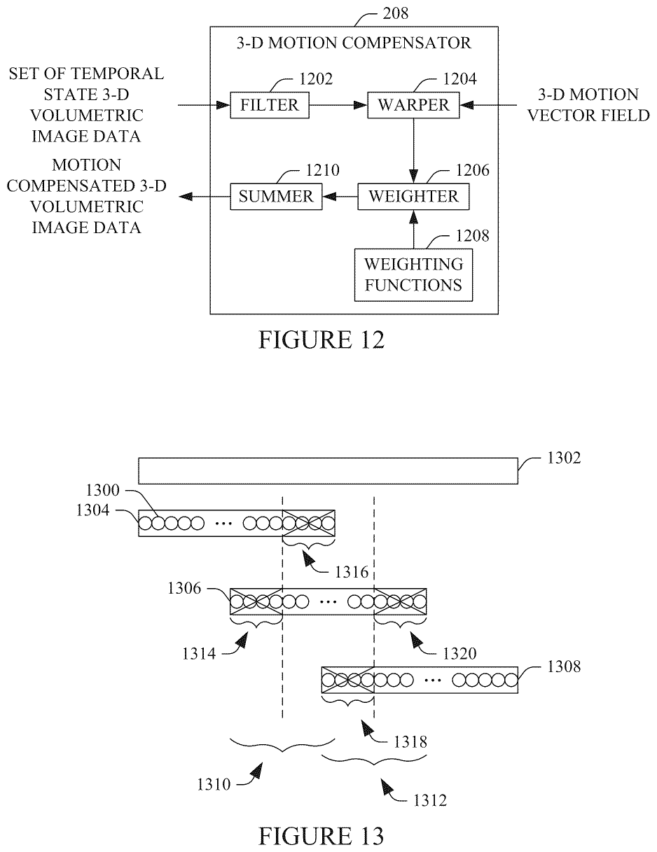

Returning to FIG. 2, the 3-D motion compensator 206 receives, as input, the at least three sets of 3-D volumetric image data (e.g., the first 3-D volumetric image data, the middle 3-D volumetric image data, and the last 3-D volumetric image data) and the 3-D MVF generated by the 3-D motion vector field determiner 204, and processes the at least three sets of 3-D volumetric image data (e.g., the first 3-D volumetric image data, the middle 3-D volumetric image data, and the last 3-D volumetric image data) using the 3-D MVF to generate motion compensated 3-D volumetric image data. Turning to FIG. 12, a non-limiting example of the 3-D motion compensator 208 is schematically illustrated. The example 3-D motion compensator 208 includes a filter 1202, a warper 1204, a weighter 1206, 3-D weighting functions 1208, and a summer 1210.

The filter 1202 is configured to divide each of the 3-D volumetric image data in the set of 3-D volumetric image data into N partial angle volumes, where N is an integer, e.g., from three (3) to thirty (30). For example, in one instance the filter 1202 includes a 2-D bowtie that divides each set of the first 3-D volumetric image data into 15 partial angle (12°) volumes. In one instance, this is achieved by rotating the bowtie 15 times in increments of 12° degrees (i.e., 180°/15) and filtering out all of the frequencies outside of the bowtie and retaining all of the frequencies inside the bowtie, which include frequencies contributed by rays perpendicular to the shape of the bowtie filter. This creates 15 partial angle (12°) volumes, each representing a different virtual time point of the scanned 3-D volume. In one instance, this is achieved via Fourier domain filtering, retaining 1/15 of the Fourier space in terms of the angular range. With three sets of 3-D volumetric image data (e.g., first, middle and last), such filtering generates 45 partial angle volumes in total, 15 for each set of 3-D volumetric image data. The bowtie-shaped filters can be tapered in the azimuthal direction. For example, there can be a total width of 24° if the azimuthal profile is triangular. In this case, each pair of two adjacent bowtie filters would overlap in a 12° range with one function dropping linearly in the azimuthal direction while the other is rising to compensate. The shape of this transition can also be a polynomial or sinusoidal shape or any other function, provided that the two functions sum to one within the overlap region.

The warper 1204 identifies a reference time point for each slice. In one instance, the reference time point for a slice is the time where the X-ray source intersects with the slice. The warper 1204 then warps each of the 45 partial angle (12°) volumes according to the estimated 3-D MVF (mm/angle) to the reference time point, using known and/or other approaches. In general, the amount of warping depends on the angular difference between the partial angle (12°) volumes and the reference time point. The weighter 1206 weights the warped partial angle (12°) volumes based on the 3-D weighting functions 1208, which are based on the above-discussed angular ranges. In one instance, the weighting weights partial angle (12°) volumes closer to the reference time point greater than partial angle (12°) volumes further away from the reference time point. In one instance, the weighting functions 1208 include smooth transitions and sum to one (1). The summer 1210 sums the weighted partial angle (12°) volumes, producing the motion compensated 3-D volumetric image data.

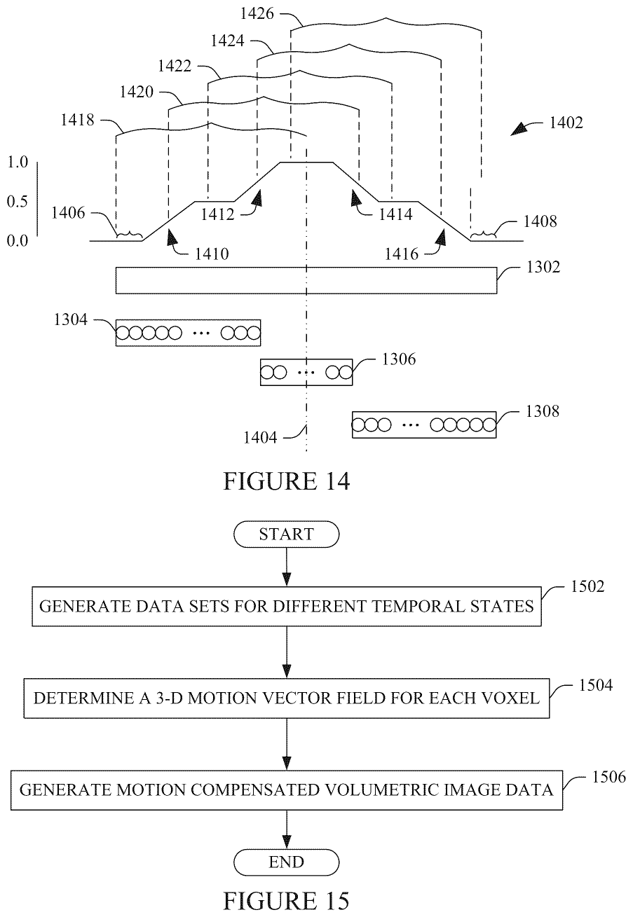

FIGS. 13 and 14 graphically illustrate an example of the weighting. For purposes of clarity and explanatory purposes, the weighting is described for a single voxel. It is to be understood that the weighting is a 3-D process and is performed for all of the voxels in the 3-D volumetric image data.

Initially referring to FIG. 13, an angular illumination range 1302 is for a particular voxel. A first angular range 1304 represents an angular range of a first 180° of the angular illumination range 1302, a middle angular range 1306 represents an angular range of a first 180° of the angular illumination range 1302, and a last angular range 1308 represents an angular range of a last 180° of the angular illumination range 1302. For each of the first angular range 1304, the middle angular range 1306 and the last angular range 1308, there are N partial angle volumes, which are graphically represented via circles 1300 (“°”) in the first angular range 1304, the middle angular range 1306 and the last angular range 1308.

The first angular range 1304 and the middle angular range 1306 overlap in an overlap region 1310. The middle angular range 1306 and the last angular range 1308 also overlap in an overlap region 1312. A region 1314 represents half of the overlap in the overlap region 1310, and a region 1316 represents a remaining half of the overlap in the overlap region 1310. A region 1318 represents half of the overlap in the overlap region 1312, and a region 1320 represents a remaining half of the overlap in the overlap region 1312.

The region 1314 of the middle angular range 1306 is removed since this region is part of the first angular range 1304 (and is better aligned with—i.e., closer to the center of—1304 than it is with 1306), the region 1320 of the middle angular range 1306 is removed since this region is part of the last angular range 1308 (and is better aligned with 1308), the region 1316 of the first angular range 1304 is removed since this region is part of (and better aligned with) the middle angular range 1306, and the region 1318 of the last angular range 1308 is removed since this region is part of (and better aligned with) the middle angular range 1306. In one instance, removal of the region 1314, the region 1316, the region 1318 and the region 1320 ensures that the data in these regions will not be utilized more than once.

Moving to FIG. 14, an example weighting function 1402 is graphically illustrated. In this example, a reference time point 1404 is approximately centered on the angular illumination range 1302 for the voxel. The illustrated weighting function 1402 has the following characteristics. There is a dead space 1406 at the beginning and a dead space 1408 at the end. The weighting function 1402 begins at a first level, has a first linear increasing transition 1410 to a second level, has another linear increasing transition 1412 to a third level, has a first linear decreasing transition 1414 back to the second level, and has another linear decreasing transition 1416 back to the first level. The weighting function 1402 is normalized such that the sum of all the values on any 180° sampling of the function is 1. In other instances, the weighting function 1402 may have similar and/or different characteristics.

The weighting function 1402 is normalized such that weights on any sampling with a spacing of 180° sum to one (1). For instance, a sum of the weights at the beginning and end of a 180° range 1418 is one (0+1), a sum of the weights at the beginning and end of a 180° range 1420 is one (0.25+0.75), a sum of the weights at the beginning and end of a 180° range 1422 is one (0.5+0.5), a sum of the weights at the beginning and end of a 180° range 1424 is one (0.75+0.25), and a sum of the weights at the beginning and end of a 180° range 1426 is one (1+0). In the above examples, there were at most two non-zero values in a 180° sampling, but if the function extends beyond a 360° range, the number of values that must sum to one can be 3 or more. In other examples, at least one pair of the transitions 1410 and 1416 and/or the transitions 1412 and 1414 is non-linear and/or there are more transitions and levels. The continuous weighting function represented in FIG. 14 is sampled at each of the angles corresponding to the circles (1300) in FIG. 13. The samples that are crossed out (or removed) are, in practice, just set to a value of zero. This process is repeated for the view weighting functions of every voxel location in the reconstruction in order to generate the weighting functions that are used by the weighter (1206).

Moving to FIG. 15, a non-limiting example of a flow chart for a computer-implemented method for generating motion compensated 3-D volumetric image data is illustrated. It is to be appreciated that the ordering of the acts in the method is not limiting. As such, other orderings are contemplated herein. In addition, one or more acts may be omitted, and/or one or more additional acts may be included.

At 1502, a set of virtual temporal state reconstructions for a helical CT (i.e., sets of 3-D volumetric image data representing different virtual time points during the helical CT scan) are generated, as described herein and/or otherwise. As described herein, the view weighting functions are configured to weight X-rays passing through each voxel at each view, where each voxel is illuminated (i.e., X-rays pass therethrough) for an angular illumination range, which is less than the angular range of the helical CT scan. In general, angular range is similar to a time range, and, therefore, reconstructions using data at different angular ranges corresponds to different virtual temporal points of the scanned volume.

In one instance, the set of virtual temporal state reconstructions includes at least three virtual temporal state reconstructions, each for a different virtual temporal state. For example, in one instance first 3-D volumetric image data is reconstructed by heavily focusing on rays at a first 180° of the angular illustration range, middle 3-D volumetric image data is reconstructed by heavily focusing on rays at a middle 180° of the angular illustration range (i.e., the target reconstruction), and last 3-D volumetric image data is reconstructed by heavily focusing on rays at a last 180° of the angular illustration range. In another instance, additional and/or other virtual temporal state reconstructions are generated.

At 1504, the filtered first 3-D volumetric image and the filtered last 3-D volumetric image data are utilized to determine the 3-D MVF between the filtered first 3-D volumetric image and the filtered last 3-D volumetric image data. In other instances, different sets of filtered 3D volumetric image are utilized, e.g., the second and fifth sets of filtered 3D volumetric image where the pitch is 0.5 and there are six reconstructions. As discussed herein, in one instance, the at least the first 3-D volumetric image and the last 3-D volumetric image data are first filtered to remove certain frequencies, e.g., to ensure that the angular gap between the centers of the two ranges is 180° or 360° and retain features suitable for motion estimation. When processing a helical scan at a low pitch, one can alternatively generate volumes that represent an angular delay of 360° instead of 180°. In one instance, an analytical approach is then utilized to iteratively determines the 3-D MVF by minimizing a difference between the moving (the filtered last 3-D volumetric image data) and fixed (the filtered first 3-D volumetric image data) reconstructions, and the 3-D MVF is converted to units of mm/angle. In another instance, a CNN or other approach is employed instead of an analytical approach.

At 1506, the 3-D MVF are utilized to generate motion compensated 3-D volumetric image data, as described herein and/or otherwise. In one instance, this includes dividing each of the sets of 3-D volumetric image into N partial angle volumes, for a total of 3N partial angle volumes in the case of three sets of 3-D volumetric image data. A reference time point is selected for each slice (i.e., the time where the X-ray source intersects with the said slice), and each partial angle volume is warped according to the estimated 3-D MVF to the reference time point. The warped partial angle volumes are recombined through weighted summation, where the weights for the partial angle volumes focus more on the partial angle volumes closer to the reference time point.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A computed tomography imaging system, comprising:
a data acquisition system configured to generate projection data indicative of X-ray radiation traversing a volume over a scan angular illumination range during a helical scan;
a reconstructor configured to reconstruct the projection data based on a set of voxel-specific weighting functions to generate at least three sets of 3-D volumetric image data, each of the at least three sets corresponding to a different virtual time point in the helical scan;
a 3-D motion vector field determiner configured to determine a 3-D motion vector field between 3-D volumetric image data corresponding to a predetermined sub-set of the different virtual time points; and
a 3-D motion compensator configured to process the at least three sets of 3-D volumetric image data based on the 3-D motion vector field to generate 3-D motion compensated 3-D volumetric image data.

2. The computed tomography imaging system of claim 1, wherein each voxel of the volume has a different set of an illumination start angle, an illumination end angle and an angular illumination range in the scan angular illumination range, and the predetermined sub-set includes:
two sets of 3-D volumetric image data angularly separated by a multiple of 180°, each corresponding to a different 180° of the scan angular illumination range.

3. The computed tomography imaging system of claim 2, wherein the two sets of 3-D volumetric image data are not initially angularly separated by the multiple of 180°, and the 3-D motion vector field determiner is further configured to:
remove overlapping angular regions in Fourier space between the two sets of 3-D volumetric image data and frequencies outside of a predetermined mid-range of frequencies, producing the two sets of 3-D volumetric image data that are angularly separated by the multiple of 180°.

4. The computed tomography imaging system of claim 2, wherein the 3-D motion vector field determiner is further configured to estimate the 3-D motion vector field based on the two sets of 3-D volumetric image data.

5. The computed tomography imaging system of claim 4, wherein the 3-D motion compensator is further configured to apply the 3-D motion vector field to the at least three sets of 3-D volumetric image data and then sum the at least three sets of 3-D volumetric image data to generate the 3-D motion compensated 3-D volumetric image data.

6. The computed tomography imaging system of claim 4, wherein the 3-D motion compensator is further configured to:
divide each of the at least three sets of 3-D volumetric image data into multiple partial angular volumes;
warp each of the partial angular volumes based on the 3-D motion vector field and a corresponding virtual time point;
weight each of the warped partial angular volumes based on a corresponding angular illumination range; and
sum the weighted warped partial angular volumes to generate the 3-D motion compensated 3-D volumetric image data.

7. The computed tomography imaging system of claim 6, wherein the 3-D motion compensator is further configured to weight overlapping angular ranges with a weight of zero.

8. A computer-implemented method, comprising:
obtaining projection data indicative of X-ray radiation traversing a volume over a scan angular illumination range during a helical CT scan;
reconstructing the projection data based on a set of voxel-specific weighting functions to generate at least three sets of 3-D volumetric image data, each of the at least three sets corresponding to a different virtual time point in the helical scan;
determining a 3-D motion vector field between 3-D volumetric image data corresponding to a predetermined sub-set of the different time points; and
processing the at least three sets of 3-D volumetric image data based on the 3-D motion vector field to generate 3-D motion compensated 3-D volumetric image data.

9. The computer-implemented method of claim 1, wherein each voxel of the voxel has a different set of an illumination start angle, an illumination end angle and an angular illumination range in the scan angular illumination range, and the predetermined sub-set includes:
two sets of 3-D volumetric image data angularly separated by a multiple of 180°, each corresponding to a different 180° of the scan angular illumination range.

10. The computer-implemented method of claim 9, wherein the two sets of 3-D volumetric image data are not initially angularly separated by the multiple of 180°, and further comprising:
removing overlapping angular regions in Fourier space between the two sets of 3-D volumetric image data and frequencies outside of a predetermined mid-range of frequencies, producing the two sets of 3-D volumetric image data that are angularly separated by the multiple of 180°.

11. The computer-implemented method of claim 10, further comprising:
estimating the 3-D motion vector field based on the two sets of 3-D volumetric image data.

12. The computer-implemented method of claim 11, further comprising:
applying the 3-D motion vector field to the at least three sets of 3-D volumetric image data; and
summing the at least three sets of 3-D volumetric image data to generate the 3-D motion compensated 3-D volumetric image data.

13. The computer-implemented method of claim 11, further comprising:
dividing each of the at least three sets of 3-D volumetric image data into a multiple partial angular volumes;
warping each of the partial angular volumes based on a corresponding portion of the 3-D motion vector field;
weighting each of the warped partial angular volumes based on a corresponding angular illumination range; and
summing the weighted warped partial angular volumes to generate the 3-D motion compensated 3-D volumetric image data.

14. The computer-implemented method of claim 13, further comprising:
removing overlapping angular ranges prior to weighting each of the warped partial angular volumes.

15. A computer readable medium encoded with computer executable instructions, which, when executed by a processor, causes the processor to:
obtain projection data indicative of X-ray radiation traversing a volume over a scan angular illumination range during a helical CT scan;
reconstruct the projection data based on a set of voxel-specific weighting functions to generate at least three sets of 3-D volumetric image data, each of the at least three sets corresponding to a different virtual time point in the helical scan;
determine a 3-D motion vector field between 3-D volumetric image data corresponding to a predetermined sub-set t of the different time points; and
process the at least three sets of 3-D volumetric image data based on the 3-D motion vector field to generate 3-D motion compensated 3-D volumetric image data.

16. The computer readable medium of claim 15, wherein each voxel of the voxel has a different set of an illumination start angle, an illumination end angle and an angular illumination range in the scan angular illumination range, and the predetermined sub-set includes:
two sets of 3-D volumetric image data angularly separated by a multiple of 180°, each corresponding to a different 180° of the scan angular illumination range.

17. The computer readable medium of claim 16, wherein the two sets of 3-D volumetric image data are not initially angularly separated by the multiple of 180°, and the computer executable instructions further cause the processor to:
remove overlapping angular regions in Fourier space between the two sets of volumetric image data and frequencies outside of a predetermined mid-range of frequencies, producing the two sets of 3-D volumetric image data that are angularly separated by the multiple of 180°.

18. The computer readable medium of claim 17, the computer executable instructions further cause the processor to:
estimate the 3-D motion vector field based on the two sets of 3-D volumetric image data.

19. The computer readable medium of claim 18, further comprising:
divide each of the at least three sets of 3-D volumetric image data into a multiple partial angular volumes;
warp each of the partial angular volumes based on a corresponding portion of the 3-D motion vector field;
weight each of the warped partial angular volumes based on a corresponding angular illumination range; and
sum the weighted warped partial angular volumes to generate the 3-D motion compensated 3-D volumetric image data.

20. The computer readable medium of claim 19, further comprising:
remove overlapping angular ranges prior to weighting each of the warped partial angular volumes.
